(19) United States
(12) Patent Application Publication
(10) Pub. No.: US 2013/0338060 A1
(43) Pub. Date: Dec. 19, 2013

(54) LIQUID MEDICINAL COMPOSITION CONTAINING ECHINOCANDIN ANTIFUNGAL AGENT MICAFUNGIN

(71) Applicants: Yunhai Hong, Shanghai (CN); Ying Xue, Shanghai (CN); Xiaoming Ji, Shanghai (CN)

(72) Inventors: Yunhai Hong, Shanghai (CN); Ying Xue, Shanghai (CN); Xiaoming Ji, Shanghai (CN)

(73) Assignee: SHANGHAI TECHWELL BIOPHARMACEUTICAL CO., LTD, Shanghai (CN)

(21) Appl. No.: 13/982,874

(22) PCT Filed: Jan. 31, 2012
(86) PCT No.: PCT/CN2012/070786

§ 371 Date: Aug. 22, 2013

(30) Foreign Application Priority Data
Jan. 31, 2011 (CN) 201110034062.0

(51) Int. Cl. A61K 47/26

(52) U.S. Cl. CPC A61K 47/26
USPC 514/3.6

(57) ABSTRACT

The present invention provides a liquid medicinal composition containing micafungin or a pharmaceutically acceptable salt thereof and a stabilizing agent.

### FIELD OF THE INVENTION

The present invention relates to a liquid pharmaceutical composition for treating and/or preventing fungal infections. Particularly, the present invention relates to the compound of Formula I or a pharmaceutically acceptable salt thereof, and a pharmaceutically acceptable amount of stabilizing agent, such as monosaccharide, disaccharide or polysaccharide, or the combinations thereof, preferably, lactose, sucrose, maltose, trehalose, or the combinations thereof. Said composition is a liquid composition.

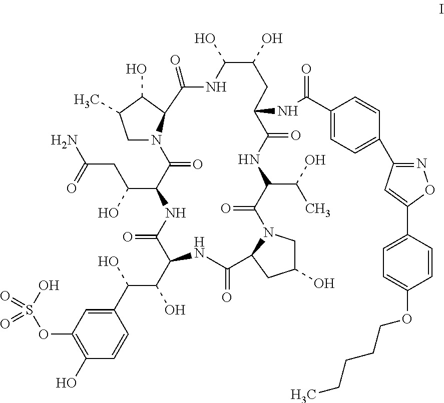

### BACKGROUND OF THE INVENTION

The compound of Formula I is cyclic polypeptide compound, i.e., Micafungin. Following Caspofungin, Micafungin Sodium (FK463, Tradename: Mycamine; Fujisawa Co.) is the second echinocandin antifungal medicament approved by FDA, the structure of which is shown in Formula III. Micafungin is obtained by chemically modifying the fermentation product of Coleophoma empedrit. Micafungin was firstly marketed in Japan in 2002, and approved by FDA and marketed in US in May 2005. Clinical test has demonstrated that Micafungin is very efficient for treating Candida and Aspergillus, and can be used as first-line medicine for treating diseases caused by Candida infections.

Since Micafungin and the salts thereof are generally unstable to light, heat, humidity, acid, and the like, it is urgent to develop a pharmaceutical composition for stabilizing the compound and the salts thereof. CN 1179748C has disclosed a stable pharmaceutical composition of Micafungin in lyophilized form comprising lactose as stabilizing agent. CN 100352495C has disclosed a stable pharmaceutical composition of Micafungin in lyophilized form comprising maltose as stabilizing agent.

However, the above pharmaceutical compositions, both of which are lyophilized preparations, are not ideal due to the complex preparation procedure, high energy consumption during lyophilization, long production cycle, limited lyophilization area of freeze dryer, all of which directly affect the production efficiency and increase the production cost. During the administration, the lyophilized preparations need to be redissolved, which will be difficult to be operated, and increase the risk of administration. Therefore, it is urgent to develop a stable pharmaceutical composition which is easy to be prepared and of low energy consumption.

The composition according to the present invention provides a safe, stable and reproducible liquid formulation, which can be directly used to treat/prevent fungal infections.

### SUMMARY OF THE INVENTION

The inventors have unexpectedly discovered that the liquid pharmaceutical composition comprising the compound of Formula I or the pharmaceutically acceptable salts thereof, and the pharmaceutically acceptable amount of stabilizing agent, such as monosaccharide, disaccharide or polysaccharide, or the combinations thereof possesses unexpected stability, the stability of which is even superior to that of the lyophilized formulations.

A pharmaceutical composition is provided by the invention, said composition comprising:

a) the echinocandin antifungal compound of Formula I or a pharmaceutically acceptable salt thereof, and

b) a pharmaceutically acceptable amount of stabilizing agent.

The composition according to the invention is a liquid formulation.

The stabilizing agent in the composition according to the present invention is monosaccharide, disaccharide or polysaccharide, or the combinations thereof, preferably, lactose, sucrose, maltose, trehalose, or the combinations thereof. The concentration of the stabilizing agent is 10-500 mg/ml, preferably, 20-400 mg/ml.

The concentration of compound of Formula I or the pharmaceutically acceptable salts thereof in the composition according to the invention is 1-150 mg/ml, preferably 5-100 mg/ml.

Preferably, the weight ratio of stabilizing agent to the compound of Formula I in the composition according to the invention is 100:1-1:20, more preferably, 20:1-1:5.

The pharmaceutical composition provided by the present invention can further comprise additional pH regulator, for example the pharmaceutically acceptable pH regulators, such as phosphate buffer, acetate buffer, citrate buffer, and the like. Preferably, pH range of the buffer is 4-7, more preferably 4.5-6.5.

In one embodiment, the pharmaceutical composition of the invention comprises the pharmaceutically acceptable salts of compound of Formula I as pharmaceutically active ingredient, and suitable, pharmaceutically acceptable amount of stabilizing agent, i.e., lactose.

In another embodiment, the pharmaceutical composition of the invention comprises the pharmaceutically acceptable salts of compound of Formula I as pharmaceutically active ingredient, and suitable, pharmaceutically acceptable amount of stabilizing agent, i.e., sucrose.

In another embodiment, the pharmaceutical composition of the invention comprises the pharmaceutically acceptable salts of compound of Formula I as pharmaceutically active ingredient, and suitable, pharmaceutically acceptable amount of stabilizing agent, i.e., maltose.

In another embodiment, the pharmaceutical composition of the invention comprises the pharmaceutically acceptable salts of compound of Formula I as pharmaceutically active ingredient, and suitable, pharmaceutically acceptable amount of stabilizing agent, i.e., trehalose.

Moreover, the composition of the invention can further comprise another, for example one or more, pharmaceutically acceptable stabilizing agent, including diluents or carriers well-known in the art, all of which are suitable for the compositions intended to be parenterally administrated, such as injectable formulations for intramuscular, subcutaneous, intravenous, intraperitoneal, or intramuscular administration. Such stabilizing agent includes, for example antioxidant, tonicity-adjusting agent, preservative, carbohydrate, wax, water-soluble and/or swellable polymer, hydrophilic or hydrophobic material, gelatin, oil, solvent, water, and the like.

Moreover, the use of the composition according to the invention is provided, for preparing medicaments, preferably intravenous medicaments for preventing and/or treating fungal infections or diseases caused by Candida and/or Aspergillus and/or Pneumocystis jirovecii in mammalian, preferably human.

As used herein, the term “Micafungin” and the pharmaceutically acceptable salts thereof have been described in U.S. Pat. No. 6,774,104B1. Preferably, the pharmaceutically acceptable salt of Micafungin is sodium Micafungin.

### DESCRIPTION OF THE DRAWINGS

FIG. 1 is the HPLC pattern for Formulation 1 in Example at 0 day under the temperature of 70° C.

FIG. 2 is the HPLC pattern for Formulation 1 in Example after stored for 4 weeks under the temperature of 70° C.

FIG. 3 is the HPLC pattern for Formulation 6 in Example at 0 day under the temperature of 70° C.

FIG. 4 is the HPLC pattern for Formulation 6 in Example after stored for 4 weeks under the temperature of 70° C.

### SPECIFIC MODE FOR CARRYING OUT THE INVENTION

HPLC method for analyzing Micafungin:

analytical column: YMC-Pack ODS-A column; spec.: 250×4.6 mm, S-5 μm, 1.2 nm;

column temperature: 35° C.;

detection: 210 nm;

mobile phase: amyl cyanide-phosphate buffer (pH 3.0) [dissolving sodium dihydrogen phosphate (16.56 g) and sodium perchlorate (7.73 g) by adding water, diluting the resulting solution to 1000 ml, and adjusting pH to 3.0 using diluted phosphoric acid (1→10)] (45:70).

The content of Micafungin was calculated according to external standard method.

All of the raw materials used in Example were produced by Shanghai Techwell Biopharmaceutical CO., LTD.

### COMPARATIVE EXAMPLE 1

### Preparation of Lyophilized Formulation of Micafungin

A composition was prepared according to Example 1 of CN 100352495C. Formulation 1 is listed as follows:

| | |
|---|---|
| Sodium Micafungin | 2.5 g |
| Lactose | 20 g |
| Anhydrous citric acid | q.s. |
| Sodium hydroxide | q.s. |

Lactose was dissolved in pure water (200 ml) with heating at the temperature less than 50° C. The lactose solution was cooled to the temperature below 20° C., and then sodium Micafungin was added with gentle agitating to avoid producing bubbles. 2% aqueous solution of citric acid (0.95 ml) was added, 0.4% aqueous sodium hydroxide solution (about 2.4 ml) was added into the resulting solution for adjusting pH to 5.5, and then the solution was diluted using pure water, thereby obtaining specified volume (250 ml). The resulting solution was loaded into 100 vials (10 ml) with each containing 2.5 ml solution. The solution in each vial was lyophilized using freeze dryer according to conventional method, thereby obtaining the lyophilized compositions each comprising 25 mg of sodium Micafungin.

The resulting lyophilized preparation was stored at 70° C., and the residue of Micafungin was tested after 4 weeks.

### COMPARATIVE EXAMPLE 2

### Preparation of Lyophilized Formulation of Micafungin

Lyophilized compositions each comprising 25 mg of sodium Micafungin (Formulation 2) were prepared according to comparative example 1, except using 15 g of maltose instead of lactose.

The same stability test was performed on the resulting lyophilized preparation as comparative example 1.

### EXAMPLE 3

### Preparation of Liquid Formulation of Micafungin

75 μl of glacial acetic acid was added into 30 ml of water, and pH of the resulting solution was adjusted to 5.5 by using 1 M NaOH. 12.0 g of trehalose was dissolved into the buffer solution, and then 1.25 g of sodium Micafungin was added. The resulting mixture was gently agitated for dissolving sodium Micafungin, water was added to obtain the specified volume (50 ml). The resulting solution was filter through 0.22 μm membrane. The composition of formulation 3 is shown in the following table:

| | |
|---|---|
| Sodium Micafungin | 25 mg/ml |
| Trehalose | 240 mg/ml |
| Glacial acetic acid | 1.5 mg/ml |
| Sodium hydroxide | Adjusting pH to 5.5 |

The prepared solution was loaded into 10 mL vials (2.5 ml/vial). All of the vials were plugged, and capped. The same stability test was performed on the resulting liquid preparations as comparative example 1.

### EXAMPLE 4

### Preparation of Liquid Formulation of Micafungin

The preparation procedure was similar to that of Example 3, except that the stabilizing agent was selected from trehalose, sucrose, lactose or maltose, and the pH regulator was selected from acetate, phosphate or citrate, even no additional pH regulator was added, thereby obtaining different formulations. The composition of each formulation is shown in the following table:

| | Sodium | | |
|---|---|---|---|
| Formulation | Micafungin, | Stabilizing agent, | pH regulator, |
| Number | mg/ml | mg/ml | pH |
| 4 | 50 | Sucrose, 200 | 25 mM phosphate, 5.5 |
| 5 | 100 | Trehalose, 200 | 10 mM citrate, 5.5 |
| 6 | 30 | Maltose, 400 | 0.4 mM citrate, 5.5 |
| 7 | 5 | Trehalose, 150 | 10 mM acetate, 5.5 |
| 8 | 1 | Lactose, 20 | [symbol] |
| 9 | 150 | Trehalose, 30 | [symbol] |
| 10 | 10 | Lactose, 80 | 0.4 mM citrate, 5.5 |
| 11 | 10 | Lactose, 10 | 0.4 mM citrate, 5.5 |

The same stability test was performed on each formulation as comparative example 1.

### EXAMPLE 5

### Preparation of Liquid Formulation of Micafungin

After the stability tests were applied to the samples from comparative example 1, comparative example 2, example 3 and example 4, the active ingredient was analyzed by HPLC.

Results for stability test of composition after stored for 4 weeks at 70° C. are shown in the following table:

| | 0 day | | | 70° C., 4 weeks | |
|---|---|---|---|---|---|
| Formulation | Appear- | Moisture, | Residue, | | Residue, |
| number | ance | % | % | Appearance | % |
| 1 | White, | 1.01 | 100 | White, | 86.1 |
| | massive | | | massive | |
| 2 | White, | 0.97 | 100 | White, | 85.7 |
| | massive | | | massive | |
| 3 | — | — | 100 | — | 94.1 |
| 4 | — | — | 100 | — | 93.7 |
| 5 | — | — | 100 | — | 94.0 |
| 6 | — | — | 100 | — | 94.3 |
| 7 | — | — | 100 | — | 94.4 |
| 8 | — | — | 100 | — | 93.8 |
| 9 | — | — | 100 | — | 93.1 |
| 10 | — | — | 100 | — | 87.1 |
| 11 | — | — | 100 | — | 85.9 |

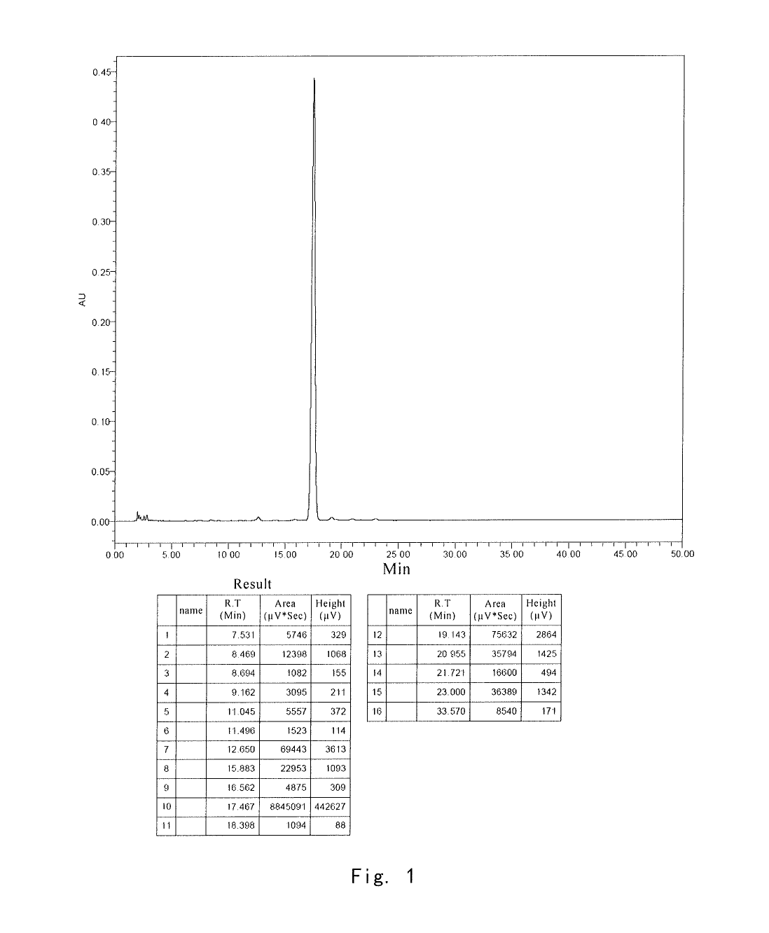

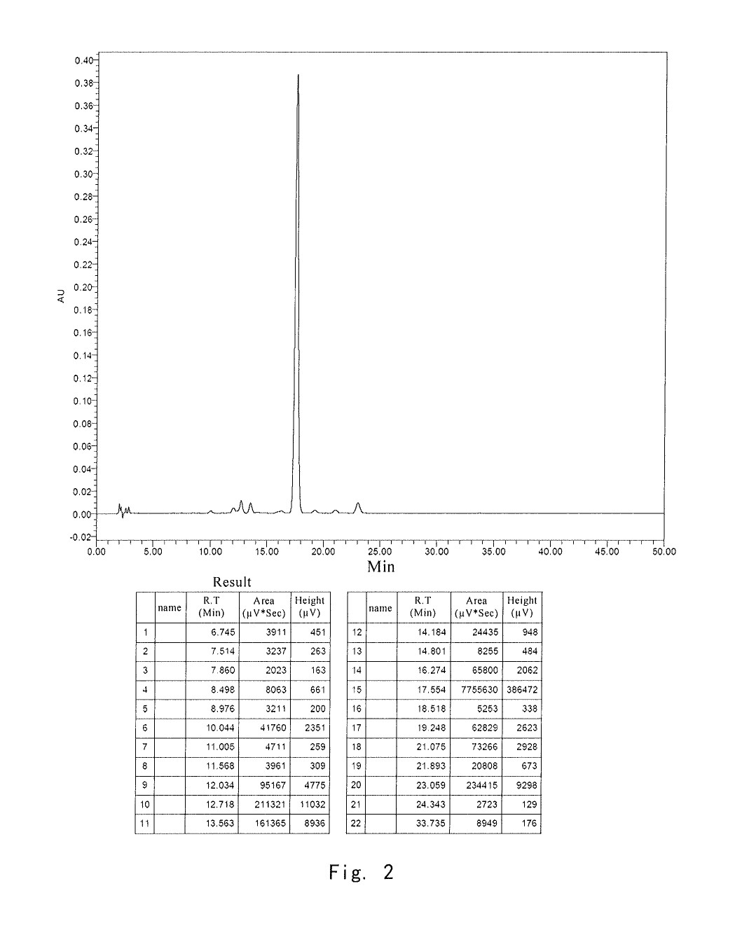

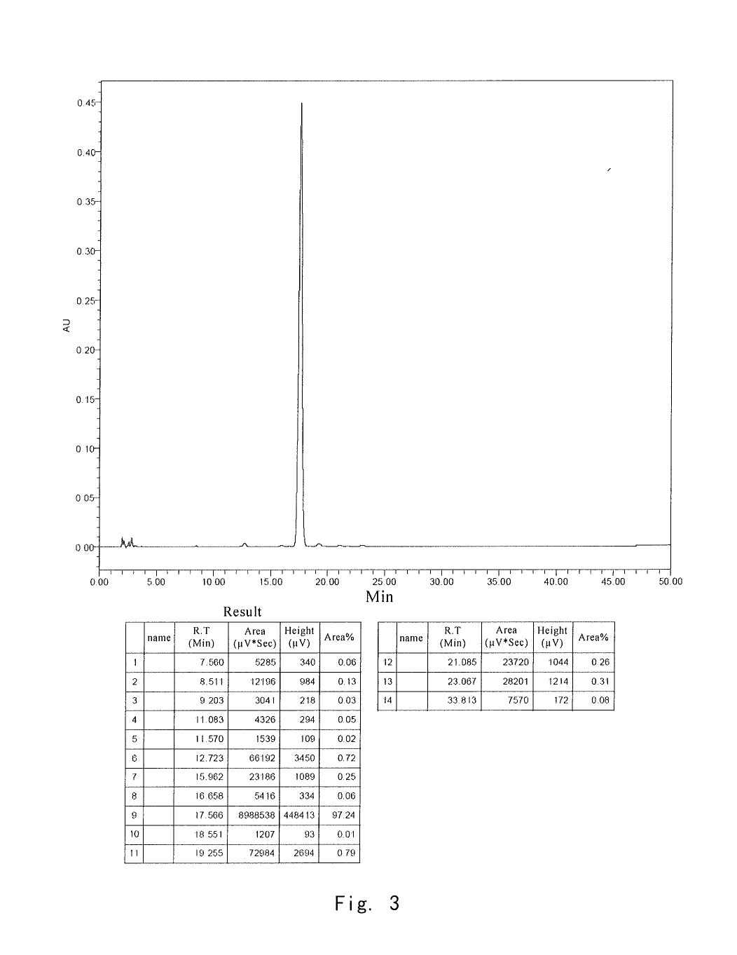

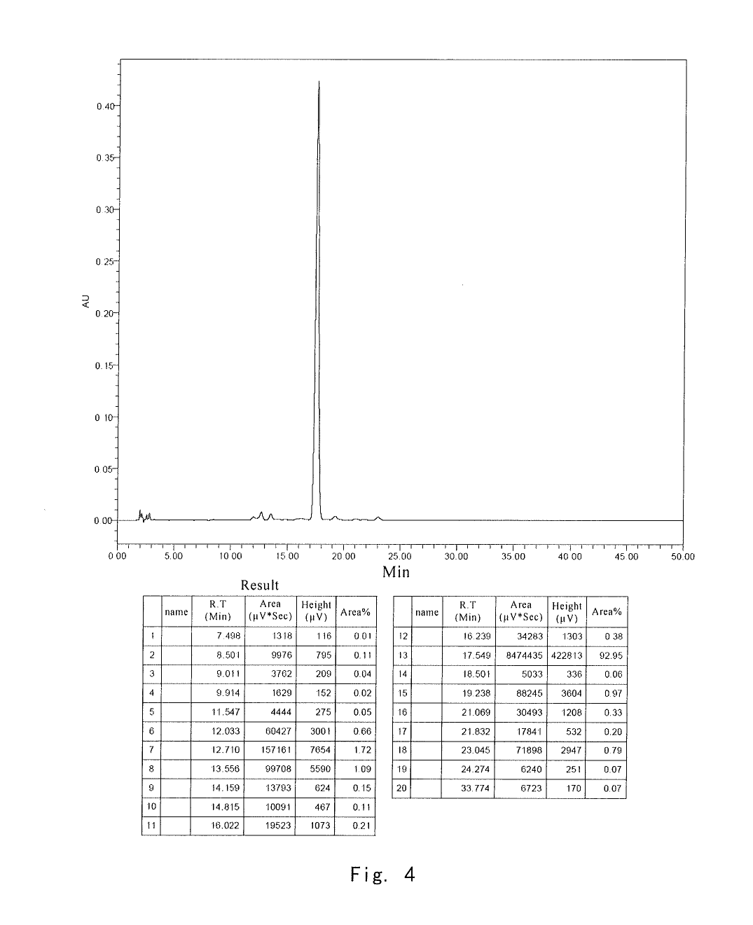

It can be seen from the above table, the liquid formulations using trehalose, sucrose, lactose or maltose or the combinations thereof as stabilizing agent with the weight ratio of stabilizing agent to sodium Micafungin being 100:1-1:20, preferably, 20:1-1:5, have excellent stability. FIGS. 1-4 show the HPLC analytical patterns for formulations 1 and 6.

1. An anti-fungi pharmaceutical composition, wherein said composition comprises:
a) a pharmaceutically efficient amount of compound of Formula I or a pharmaceutically acceptable salt thereof; and
b) a pharmaceutically acceptable amount of stabilizing agent

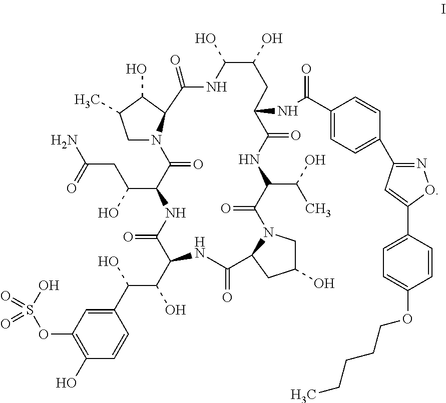

2. The pharmaceutical composition according to claim 1, wherein preferably, the pharmaceutically acceptable salt of compound of Formula I is sodium salt.

3. The pharmaceutical composition according to claim 1, wherein said stabilizing agent is selected from: monosaccharide, disaccharide or polysaccharide, or the combinations thereof; preferably, lactose, sucrose, maltose, trehalose, or the combinations thereof.

4. The pharmaceutical composition according to claim 1, wherein said composition is a liquid formulation.

5. The pharmaceutical composition according to claim 1, wherein the composition contains 1-150 mg/ml of compound of Formula I or a pharmaceutically acceptable salt thereof.

6. The pharmaceutical composition according to claim 5, wherein the composition contains 5-100 mg/ml of compound of Formula I or a pharmaceutically acceptable salt thereof.

7. The pharmaceutical composition according to claim 1, wherein the composition contains 10-500 mg/ml of stabilizing agent.

8. The pharmaceutical composition according to claim 7, wherein the composition contains 20-400 mg/ml of stabilizing agent.

9. Use of the pharmaceutical composition according to claim 1, wherein the composition is used for preparing medicaments for preventing and/or treating fungal infections in mammalian.
